(19) United States
(12) Patent Application Publication
(10) Pub. No.: US 2022/0334050 A1
(43) Pub. Date: Oct. 20, 2022

(54) ADAPTER

(71) Applicant: Loewenstein Medical Technology S.A., Luxembourg (LU)

(72) Inventor: Peter Kremeier, Karlsruhe (DE)

(21) Appl. No.: 17/724,255

(22) Filed: Apr. 19, 2022

(30) Foreign Application Priority Data
Apr. 20, 2021 (DE) 102021002052.9

(51) Int. Cl. G01N 21/05

(52) U.S. Cl. CPC G01N 21/05; G01N 2021/052

(57) ABSTRACT

An adapter for use with spectrometers for the analysis of gases, comprising a housing which comprises a base body, an accommodator for accommodation of a spectrometer, a housing entrance and a housing exit, the housing entrance comprising an inlet opening and the housing exit comprising an outlet opening, a passage channel being arranged between the inlet opening and the outlet opening, and the base body being arranged between the housing entrance and the housing exit. The base body has at least two openings which are arranged on two sides of the passage channel on opposite sides.

### CROSS-REFERENCE TO RELATED APPLICATIONS

The present application claims priority under 35 U.S.C. § 119 of German Patent Application No. 102021002052.9, filed Apr. 20, 2021, the entire disclosure of which is expressly incorporated by reference herein.

### BACKGROUND OF THE INVENTION

### 1. Field of the Invention

The invention relates to an adapter for use with spectrometers, for example infrared spectrometers (IR spectrometers), for the analysis of gases.

### 2. Discussion of Background Information

Adapters for use with IR spectrometers are used in the field of medical technology, for example in the field of ventilation. The adapter can also be referred to as a cuvette. The terms adapter and cuvette are used herein as synonyms.

The analysis of breathing gases can be carried out such that the air exhaled by a patient is conducted by an adapter. The adapter is usually equipped with windows for the passage of an IR or light beam, which consist of light- and/or infrared-transparent materials. In medicine, use is made of disposable and reusable adapters for mainstream infrared spectrometers. Reusable adapters must withstand various kinds of sterilization and must not lose their properties at the same time.

For reusable adapters, use is usually made of sapphire windows, which exhibit high stability and are transparent for the infrared region of the electromagnetic spectrum. Plastic windows are generally less stable and change their properties during sterilization. Therefore, plastic windows are only used in disposable adapters.

Various kinds of sterilization can be used for sterilizing reusable adapters, especially thermal (steam or dry heat) or chemical sterilization. These are the most common kinds of sterilization and have the most damaging effects on the material of the device.

The action of steam and high temperature leads to weakening and destruction of adhesive and other kinds of joints between different materials. The effect of thermal expansion is greater if the joint contains more than one kind of material. In combination with high temperature and high pressure, steam penetrates into the adhesive layer and changes the properties thereof, which adversely affects the integrity of the adapter and impenetrability.

Chemical sterilization is carried out using aggressive solutions, which also damage adhesive compounds. In this respect, the attachment of the window to the adapter housing is a critical step in the production process, especially in cases in which the adapter is made of a different material than the window.

JP 3677672 B2, the entire disclosure of which is incorporated by reference herein, proposes an adapter construction for measurement of the concentration of gas components by the spectrometric method, which consists in preventing the formation of droplets of condensed liquid from the airstream on the inner window surface together with a transparent window frame. The disadvantage of this technical solution is the change in cross-sectional area of the airway of the cuvette, which subsequently leads to an increase in the concentration rise time.

US 2016/0184545 A1, the entire disclosure of which is incorporated by reference herein, discloses an adapter casting process which makes it possible to obtain an adapter which contains a path for an airstream and integrated windows allowing measurements of the airstream through the adapter. The casting is done in one step, with the required window thickness, which is lower than the normal thickness of the adapter walls, being achieved by using pins which press the previously shaped window surface to the required thickness. The windows are located on opposite sides of the airway of the adapter.

Adapter and windows form a one-piece construction, and the windows consist of the same material as the housing of the adapter. In this respect, the optical properties of the window correspond to the optical properties of the housing material. In most cases, it is not appropriate to produce the device from the same material, since this leads to additional costs. With regard to production, it is assumed that the device housing consists of polymeric plastics. However, the optical properties of such windows are poorer than the optical properties of, for example, sapphire windows.

U.S. Pat. No. 7,629,039 B2, the entire disclosure of which is incorporated by reference herein, proposes a window construction for use in an adapter for an infrared gas analyzer for analysis of exhaled air, the gas flowing through the passage in the adapter having a window located on mutually opposing sides of the channel, so that an infrared beam can be directed through the window and the channel containing the specified exhaled gas. The window is produced in the form of a solid plastic structure and has a substantially circular shape and comprises a surrounding edge and a central part, sunken in relation to the specified edge and forming a window through which the passage of infrared beams is ensured.

The disadvantage of this technical solution is the presence of a cavity between the sunken window of the adapter and the gas analyzer, which may contain carbon dioxide due to accidental exhalation by medical personal when fitting the adapter into the gas analyzer. This carbon dioxide increases an error in the measurement result.

In view of the foregoing, it would be advantageous to have available an adapter which is simple and cost-effective to produce and nevertheless delivers reliable measurement results.

### SUMMARY OF THE INVENTION

The present invention provides an adapter for use with spectrometers for the analysis of gases, having a housing which comprises a base body, an accommodator for accommodation of a spectrometer, a housing entrance and a housing exit, the housing entrance comprising an inlet opening and the housing exit comprising an outlet opening, a passage channel being arranged between the inlet opening and the outlet opening, and the base body being arranged between the housing entrance and the housing exit. The base body has at least two openings which are arranged on two sides of the passage channel on opposite sides.

In some embodiments, the adapter is characterized in that the base body has two side walls which each have an opening through which radiation can be directed such that the radiation intersects the passage channel

In some embodiments, the adapter is characterized in that the radiation is infrared radiation.

In some embodiments, the adapter is characterized in that the passage channel is designed and configured to guide breathing gases.

In some embodiments, the adapter is characterized in that the adapter has a maximum length, wherein the passage channel extends from the inlet opening to the outlet opening over the maximum length of the adapter.

In some embodiments, the adapter is characterized in that the openings are a polygon, preferably a quadrangle, in terms of their basic shape.

In some embodiments, the adapter is characterized in that at least one of the at least two openings is covered by at least one cover to establish an operating mode.

In some embodiments, the adapter is characterized in that adapter and cover are in the form of one piece or two pieces.

In some embodiments, the adapter is characterized in that adapter and cover consist of the same material or of different materials.

In some embodiments, the adapter is characterized in that adapter and cover are in the form of two pieces and made of the same material.

In some embodiments, the adapter is characterized in that the adapter is made of a plastic, preferably of a thermoplastic.

In some embodiments, the adapter is characterized in that the adapter is made of a polycarbonate (PC), a polymethyl methacrylate (PMMA), a polystyrene (PS) or a cycloolefin copolymer (COC, Topas).

In some embodiments, the adapter is characterized in that the adapter is made of one or more of PMMA Plexiglas 7N, Topas 6017-S04, PC Lexan 121 RM1, PC Makrolon 2400, PS Styrolution 124 L, Topas 8007 or COC 8007 X-04, preferably of COC or PMMA.

In some embodiments, the adapter is characterized in that the cover is transmissive for radiation, especially IR radiation. In some embodiments, the adapter is characterized in that the cover has a transmissivity for infrared radiation of at least 0.5.

In some embodiments, the adapter is characterized in that the cover is made of a plastic.

In some embodiments, the adapter is characterized in that the cover is made of a polycarbonate (PC), a polymethyl methacrylate (PMMA), a polystyrene (PS) or a cycloolefin copolymer (COC, Topas).

In some embodiments, the adapter is characterized in that the cover is made of one or more of PMMA Plexiglas 7N, Topas 6017-S04, PC Lexan 121 RM1, PC Makrolon 2400, PS Styrolution 124 L, Topas 8007 or COC 8007 X-04, preferably of COC 8007 X-04.

In some embodiments, the adapter is characterized in that the cover is a film.

In some embodiments, the adapter is characterized in that the film has a thickness from about 2,000 μm to about 50 μm, preferably from about 1,000 μm to about 100 μm, particularly preferably from about 600 μm to about 100 μm, for example about 140 μm.

In some embodiments, the adapter is characterized in that the film is larger than the opening.

In some embodiments, the adapter is characterized in that the base body comprises two side walls which each comprise an opening, an inner edge, an outer edge and a side face.

In some embodiments, the adapter is characterized in that the film is applied to the side walls at least regionally and completely covers at least one of the at least two openings to establish an operating mode of the adapter.

In some embodiments, the adapter is characterized in that the film covers the openings such that the passage channel is sealed in an air-tight manner and passage of gases is solely possible from the housing entrance to the housing exit and vice versa.

In some embodiments, the adapter is characterized in that the film is joined to the side wall or portions of the side wall by adhesive bonding, heat sealing and/or mechanical latching.

In some embodiments, the adapter is characterized in that from about 20% to about 80% of the film are joined to the side wall or to portions of the side wall, preferably from about 30% to about 60%, particularly preferably from about 40% to about 50%.

In some embodiments, the adapter is characterized in that the film is joined to the inner edge and/or the outer edge and/or the side face at least regionally.

In some embodiments, the adapter is characterized in that the film and the inner edge and/or the outer edge and/or the side face are joined by means of laser welding.

In some embodiments, the adapter is characterized in that the film has a polygonal shape, preferably a quadrangular shape.

In some embodiments, the adapter is characterized in that the film comprises side lengths and at least one rounded corner, preferably two rounded corners.

In some embodiments, the adapter is characterized in that the side lengths of the film are straight or curved, preferably straight.

In some embodiments, the adapter is characterized in that the side lengths of the film have a length of from about 5 mm to about 60 mm, preferably from about 10 mm to about 30 mm, particularly preferably from about 15 to about 20 mm.

In some embodiments, the adapter is characterized in that the accommodator for accommodation of a spectrometer is adjacent to the base body.

In some embodiments, the adapter is characterized in that the accommodator is integral with the housing.

In some embodiments, the adapter is characterized in that the accommodator comprises an accommodation base and two accommodation side walls, within which the base body is arranged.

In some embodiments, the adapter is characterized in that the accommodation side walls and the accommodation base are broader than the base body is deep. In some embodiments, the adapter is characterized in that the accommodation side walls are taller than the base body is tall.

In some embodiments, the adapter is characterized in that the accommodator is configured and designed to accommodate a shape-complementary IR spectrometer between the two accommodation side walls and the accommodation base such that at least the at least two openings of the base body are enclosed by the IR spectrometer.

In some embodiments, the adapter is characterized in that, upon accommodation of the IR spectrometer in the accommodator, IR radiation can be directed through the openings covered by at least one film such that the IR radiation intersects the passage channel

In some embodiments, the adapter is characterized in that at least one accommodation side wall comprises at least one accommodation latch, wherein the accommodation latch is configured and designed to fix the IR spectrometer in a measurement position.

The present invention also provides a system for analysis of a stream of breathing gas, at least comprising a ventilator and/or a patient interface and an IR spectrometer, wherein the system comprises at least one adapter as set forth above (including the various embodiments thereof), wherein the adapter is gaseously connected to the ventilator and/or the patient interface and wherein the IR spectrometer encloses the adapter at least regionally such that IR radiation intersects the stream of breathing gas.

In some embodiments, the system is characterized in that the adapter is arranged in and/or on the ventilator and/or in and/or on the patient interface.

In some embodiments, the system is characterized in that the adapter is connected to the ventilator and/or the patient interface via at least one line.

### BRIEF DESCRIPTION OF THE DRAWINGS

The drawings show exemplary, non-limiting embodiments of the adapter according to the invention, in which:

FIG. 1 shows an overall view of an adapter according to the invention for use with spectrometers for the analysis of gases, for example of breathing gases, in plan view from the side;

FIG. 2 shows a perspective view of an adapter according to the invention;

FIG. 3 shows an adapter according to the invention in plan view from the side

FIG. 4 shows an adapter according to the invention in plan view from the side, with additional depiction of covers (A), which are applied to the adapter to establish an operating mode (B), and an IR spectrometer 80 located on the operational adapter 10 in a measurement position (C);

FIG. 5 shows a film for an adapter according to the invention;

FIG. 6 shows an adapter according to the invention in plan view from the top;

FIG. 7 shows an adapter according to the invention in plan view from the bottom;

FIG. 8 shows a longitudinal section according to line G-G through an adapter according to the invention;

FIG. 9 shows an adapter according to the invention in plan view from the housing entrance side;

FIG. 10 shows an adapter according to the invention in plan view from the housing exit side;

FIG. 11 shows a cross section according to line B-B, which essentially runs through the base body of an adapter according to the invention;

FIG. 12 shows a detailed view of the section according to line B-B in the region Y;

FIG. 13 shows a detailed view of the section according to line B-B in the region X; and

FIG. 14 shows a schematic overall view of a system comprising an adapter according to the invention having a mounted IR spectrometer that is gaseously connected to a ventilator and a patient interface via a line.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

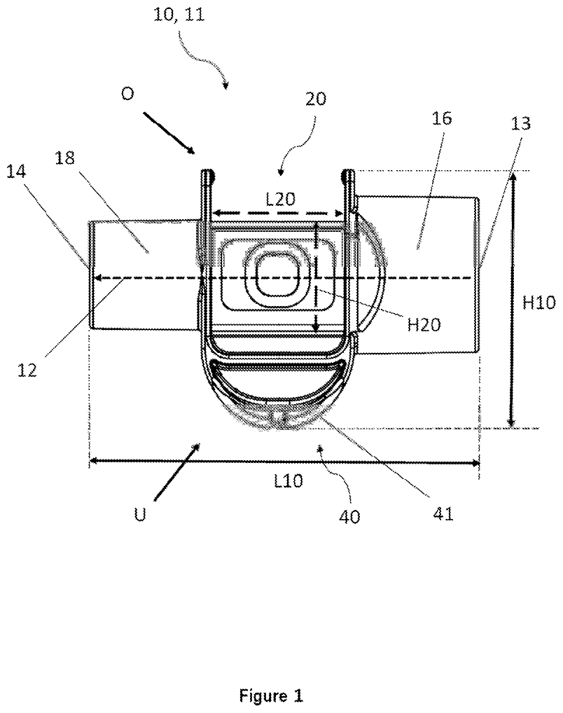

FIG. 1 shows an overall view of an adapter 10 according to the invention for use with spectrometers for the analysis of gases, for example of breathing gases, in plan view from the side. The adapter 10 can also be referred to as a cuvette or flow cuvette.

The adapter 10 is configured and designed to receive gases such that they can be analyzed. For the analysis, use is made of a spectrometer, for example an infrared spectrometer (IR spectrometer) 80 (not shown) that is complementary in shape to the adapter 10. Using the IR spectrometer 80, it is possible to determine, for example, the CO₂ content of a breathing gas. Other quantitative or qualitative analyses are likewise conceivable.

The adapter 10 is configured and designed such that infrared radiation (IR radiation) can be guided through the adapter 10 such that the radiation intersects the breathing gas to be analyzed in the adapter 10.

The adapter 10 is, for example, made of a plastic. In particular, the adapter 10 is made of a thermoplastic. The adapter 10 can, for example, be produced by means of an injection molding process. However, also conceivable are other suitable materials and production processes that are suitable with regard to manufacturing properties, costs, weight, weldability and temperature resistance.

Suitable plastics encompass polycarbonates (PC), polymethyl methacrylates (PMMA), polystyrenes (PS) or cycloolefin copolymer (COC, Topas). For example, the adapter 10 is selected from the group consisting of PMMA Plexiglas 7N, Topas 6017-S04, PC Lexan 121 RM1, PC Makrolon 2400, PS Styrolution 124 L, Topas 8007 or COC 8007 X-04. Preferably, the adapter 10 is made of COC 8007-04.

The adapter 10 can have at least one grip 41. By definition, the side on which the grip 41 is located is the bottom side or bottom (U). The side which is opposite the grip 41 is, by definition, the top side or top (O).

The adapter 10 comprises a housing 11 and a passage channel 12.

The adapter 10 has a maximum length L10 and a maximum height H10. The length L10 is generally greater than the height H10. However, length L10 and height H10 can also be identical and the height H10 can also be greater than the length L10. The passage channel 12 extends through the entire length L10 of the adapter 10.

The adapter 10 has at least one inlet opening 13 and one outlet opening 14. The passage channel 12 extends from the inlet opening 13 to the outlet opening 14. The passage channel 12 is configured and designed to guide gases, especially breathing gases. The breathing gas can be conducted into the adapter 10 and/or flow therethrough along the passage channel 12.

In the exemplary embodiment, the housing 11 comprises essentially four components, namely a housing entrance 16, a housing exit 18, an accommodator 40 and a base body 20.

The accommodator 40 is arranged between the housing entrance 16 and the housing exit 18. The base body 20 is arranged adjacent to the accommodator 40. View from the side, the accommodator 40 has essentially a U-shape. The base body 20 is located within the U-shape. The base body 20 is therefore enclosed by the accommodator 40 by means of the U-shape.

Accommodator 40 and base body 20 are configured and designed to accommodate a shape-complementary IR spectrometer 80 such that at least portions of the base body 20 are enclosed by the IR spectrometer 80.

Connections can be connected to the housing entrance 16 and housing exit 18. For example, what can be connected are hoses which form a connection to a gas source and/or to a patient interface (not shown). The breathing gas can therefore be conducted into the adapter 10 via the housing entrance 16 and be guided out of the adapter 10 via the housing exit 18 and vice versa. The breathing gas is then located in the passage channel 12 and/or then passes therethrough.

Advantageously, the adapter 10 is of a compact construction. This can minimize the volume of the passage channel 12.

The entire length L10 of the adapter 10 is not more than 120 mm, preferably less than 80 mm. In one exemplary embodiment, the length L10 of the adapter 10 is within a range of 70 mm to 30 mm. The length L10 of the adapter 10 is, for example, 55.6 mm.

In some embodiments, housing entrance 16 and housing exit 18 can, for example, have smaller dimensions, which reduces the entire length L10 of the adapter 10. In other embodiments, housing entrance 16 and housing exit 18 can also be longer, and so the entire length L10 of the adapter increases. The length of the passage channel 12 is directly dependent on the length L10.

The height H10 of the adapter 10 is not more than about 120 mm, preferably less than about 80 mm. In one exemplary embodiment, the height H10 of the adapter 10 is within a range of from about 60 mm to about 20 mm. The height H10 of the adapter 10 is, for example, 36.75 mm.

In the present embodiment, the height H10 of the adapter 10 is determined by the dimensions of the accommodator 40 and the grip 41. For example, an alternative embodiment of the grip 41 can increase or reduce the total height H10 of the adapter 10. An alternative embodiment of the accommodator 40 can likewise increase or reduce the total height H10 of the adapter. The passage channel 12 is not directly dependent on the height H10 of the adapter 10 in the present embodiment.

The volume of the passage channel is dependent on the length L10 of the adapter 10. In the present embodiment, the passage channel 12 holds a volume of 6.6 ml. What is ideal is a smallest possible volume from about 0.5 ml to about 7 ml depending on the category of patient.

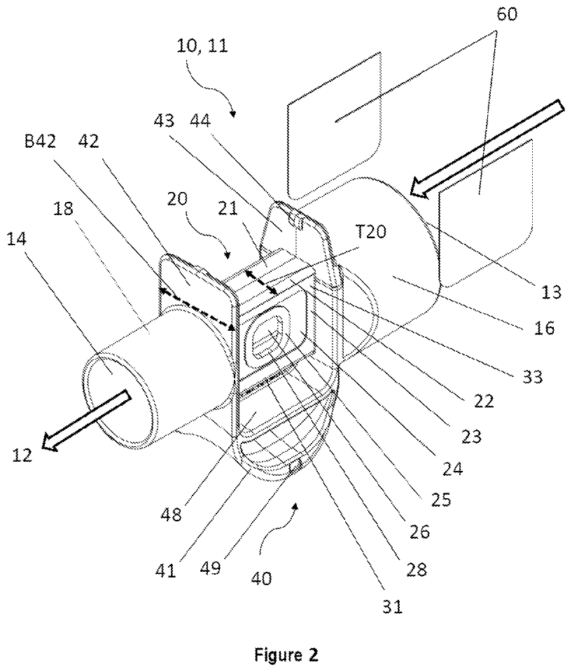

The base body 20 is a further component of the four essential components of the adapter 10. The base body 20 has a height H20, a length L20 and a depth T20 (see FIGS. 1 and 2). In the present exemplary embodiment, the length L20 is greater than the height H20. Length L20 and height H20 can also be identical and the height H20 can, in other exemplary embodiments, also be greater than the length L20.

The gas analysis takes place in the base body 20. For this purpose, the base body 20 should have a smallest possible volume, but at the same time provide no appreciable flow resistance. The dimensions of the base body 20 can be optimized depending on the nature and number of measurements.

The length L20 of the base body 20 is from about 60 mm to about 4 mm, preferably from about 30 mm to about 10 mm, even more preferably from about 21 mm to about 15 mm. The length L20 of the base body 20 is, for example, from 19.3 to 19.5 mm. The height H20 of the base body 20 is from about 60 mm to about 4 mm, preferably from about 30 mm to about 10 mm, even more preferably from about 16 mm to about 13 mm. The height H20 of the base body 20 is, for example, from 15 mm to 15.2 mm. The depth T20 of the base body 20 is from about 60 mm to about 2 mm, preferably from about 20 mm to about 4 mm. The depth T20 of the base body 20 is, for example, 7.55 mm (see FIG. 2).

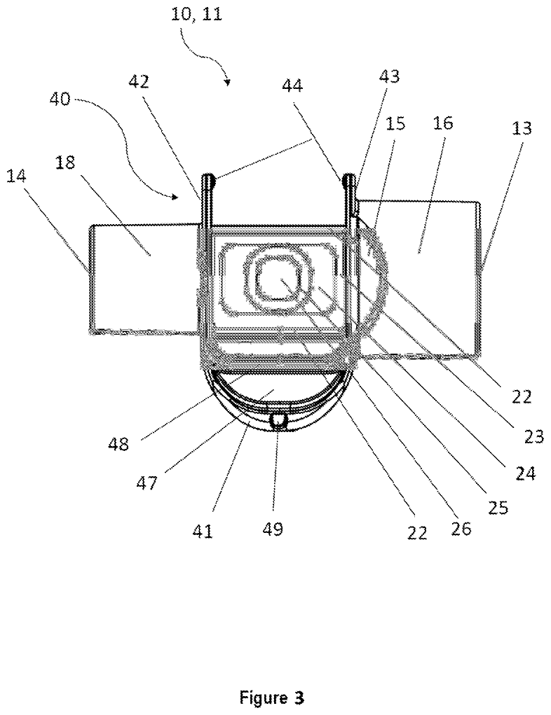

FIG. 2 shows a perspective view of an adapter 10 according to the invention. FIG. 3 shows an adapter 10 according to the invention in plan view from the side.

FIG. 2 depicts the housing 11 with the four components housing entrance 16, housing exit 18, accommodator 40 and base body 20 in one exemplary embodiment according to the invention. The housing entrance 16 and the housing exit 18 are in the form of an elongated hollow body. Housing entrance 16 and housing exit 18 generally have a circular cross section. An oval or angular shape is also possible. Housing entrance 16 and housing exit 18 are, for example, in the form of an elongated hollow body having a circular cross section. Housing entrance 16 and housing exit 18 can also be referred to as connecting pieces. In the present exemplary embodiment, the housing entrance 16 has a larger diameter than the housing exit 18. Other possibilities are that housing entrance 16 and housing exit 18 are of identical diameter and that the diameter of the housing exit 18 is larger than that of the housing entrance 16.

The housing entrance 16 comprise the inlet opening 13. It is through the inlet opening 13 that gases can be conducted into the adapter 10. The inlet opening 13 represents the start of the passage channel 12. The gases therefore get into the passage channel 12 through the inlet opening 13.

The housing exit 18 comprises the outlet opening 14. It is through the outlet opening 14 that gases can be conducted out of the adapter 10. The outlet opening 14 represents the end of the passage channel 12. The gases are therefore generally conducted into the passage channel 12 through the inlet opening 13 and conducted out of the passage channel 12 through the outlet opening 14. A reverse gas flow is possible, too.

Located between the housing entrance 16 and the housing exit 18 is the accommodator 40 and the base body 20.

The base body 20 is in the form of a hollow body. The base body 20 is, for example, in the form of a square tube having four plane-parallel faces. The four faces of the base body 20 are preferably substantially perpendicular to one another. The passage channel 12 passes through the base body 20.

The base body 20 comprises a top side 21, a bottom side 31 and two side walls 33. The top side 21 and the bottom side 31 define a depth of the base body T20. The side walls 33 define a height of the base body H20 and a length of the base body L20 (see FIG. 1).

Top side 21 and bottom side 31 are parallel to one another. Top side 21 and bottom side 31 are, for example, planar and closed. The edges of top side 21 and bottom side 31 to the side walls 33 can be sharp-edged. In the exemplary embodiment shown, the sharp edges have been bevelled by a chamfer 22.

The side walls 33 are parallel to one another. FIG. 2 merely depicts one side wall 33; in the present exemplary embodiment, the corresponding second side wall 33 is mirror-symmetrical. Differently designed side walls 33 are also conceivable. Differently designed side walls 33 are, for example, conceivable in order to accommodate an IR spectrometer 80 in a predetermined orientation and to thus specify a direction of measurement.

The side walls 33 each comprise at least one opening 26,27, each having a reveal 28. Multiple openings are also conceivable in order, for example, to be able to carry out various measurements in parallel. In the present exemplary embodiment, the adapter 10 has two openings 26, 27, only one of which is depicted in FIGS. 2 and 3. The openings 26, 27 are identical. The openings 26, 27 are a polygon in terms of their basic shape. In this exemplary embodiment, the openings 26, 27 form a quadrangle. The corners of the polygon can be angular or rounded. In this exemplary embodiment, the openings 26, 27 are, for example, in the form of a square having rounded corners.

Each opening has a reveal 28. The reveal 28 is perpendicular to the side wall 33. The reveal 28 is the inner wall face of the side wall 33 that is facing the openings 26, 27. The reveal 28 completely comprises the opening 26, 27. The reveal 28 preferably has a constant depth.

In a simple embodiment, the side walls 33 only contain the openings 26, 27 and the reveal 28. In this case, the depth of the reveal 28 corresponds to the thickness of the side wall 33. In the embodiment depicted in the figures, the side walls 33 comprise not only the opening 26, 27 and the reveal 28 but also, in each case, an inner edge 25, a side face 24 and an outer edge 23.

The inner edge 25 completely surrounds the opening 26, 27. The inner edge 25 can have any suitable shape. The inner edge 25 can, for example, have a shape corresponding to the opening 26, 27. The inner edge 25 is, for example, in the form of a square frame having rounded corners. In this example, the depth of the reveal 28 corresponds to the thickness of the inner edge 25. The reveal 28 is from about 0.4 mm to about 4 mm in depth. The reveal 28 is, for example, 0.9 mm in depth. The reveal 28 should have a smallest possible depth in order to avoid turbulences of the gas flow in the passage channel 12 and to minimize measurement errors.

The outer edge 23 outwardly delimits the side wall 33. The outer edge 23 has a rectangular shape. The outer edge 23 corresponds to the shape of the side wall 33. In the present exemplary embodiment, the outer edge 23 is accordingly greater in its breadth than in its height. In the present exemplary embodiment, the inner edge 25 and the outer edge 23 have an identical elevation profile.

In the present exemplary embodiment, a side face 24 is arranged between the inner edge 25 and the outer edge 23. The side face is slightly indented in comparison with the inner edge 25 and with the outer edge 23. The indentation is from about 0.1 mm to about 3 mm, for example 0.5 mm. In the present exemplary embodiment, the side face 24 is rectangular and is greater in its breadth than in its height.

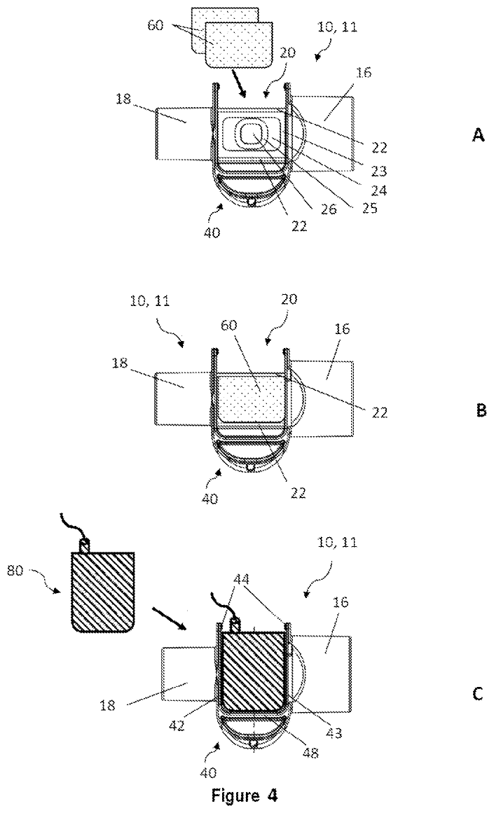

Owing to the mirror-symmetry of the side faces 24, the openings 26 and 27 are located on a mutually corresponding position within the side faces 24. The openings 26, 27 are therefore arranged on two sides of the passage channel 12 on opposite sides. Upon accommodation of a shape-complementary IR spectrometer 80, the base body 20 is enclosed by the IR spectrometer 80 (see FIG. 4C) in such a way that IR radiation can be directed through the openings 26, 27 such that the IR radiation intersects the breathing gas present in the base body 20.

The fourth component of the adapter 10 is the accommodator 40. The accommodator 40 is configured and designed to accommodate a shape-complementary IR spectrometer 80 and to fix it in a measurement position.

View from the side, the accommodator 40 has essentially a U-shape (see FIG. 3). The accommodator 40 is arranged adjacent to the base body 20. The base body 20 is enclosed by the accommodator 40 by means of the U-shape.

The accommodator 40 comprises two accommodation side walls 42, 43 having at least one, preferably two accommodation latches 44 and one accommodation base 48.

The first accommodation side wall 42 is arranged adjacent to the base body 20 and to the housing exit 18. The second accommodation side wall 43 is arranged adjacent to the base body 20 and to the housing entrance 16. The accommodation side walls 42, 43 are arranged perpendicular to the side walls 33 and to the top side 21 and the bottom side 31 of the base body 20. The accommodation side walls 42,43 are arranged on the adapter 10 in a saddle-like manner

The accommodation side walls 42,43 are larger than the base body 20. The accommodation side walls 42, 43 are broader than the base body 20 is deep. The accommodation side walls 42, 43 are taller than the base body 20 is tall. The accommodation side walls 42, 43 therefore project beyond the depth of the base body T20 and beyond the height of the base body H20.

At the bottom end, the accommodation side walls 42, 43 are connected to one another via an accommodation base 48. The accommodation side walls 42, 43 are, for example, perpendicular on the accommodation base 48. It is also conceivable that the accommodation side walls 42,43 are arranged on the accommodation base 48 in an outwardly leaning manner At the top end, the accommodation side walls 42, 43 are not connected. This gives rise to a U-shape.

The transition from the accommodation side walls 42, 43 to the accommodation base 48 can be angular. As depicted in FIGS. 2 and 3, the transition can also have rounded corners. The accommodation base 48 is, for example, planar. The accommodation base 48 can also be rounded. The accommodation base 48 is broader than the base body 20 is deep. The accommodation base 48 is therefore also broader than the bottom side 31. The accommodation base 48 is, for example, just as broad as the accommodation side walls 42, 43 (see FIG. 2).

Because both the accommodation side walls 42, 43 and the accommodation base 48 are broader than the base body 20 is deep, what are provided are free faces on which a shape-complementary IR spectrometer 80 can abut (see herein further below, FIG. 4C). Moreover, the accommodation side walls 42, 43 and the accommodation base 48 offer shadowing of the base body, which is advantageous during measurement using the IR spectrometer 80. The accommodator 40 shields the surrounding radiation. This reduces or avoids measurement errors due to the influence of ambient radiation during spectrometric measurement.

The adapter 10 can comprise a grip 41. The grip 41 is arranged below the accommodation base 48. The grip 41 can have any suitable shape. In the present exemplary embodiment, the grip 41 has a semicircular shape.

In the case of production of the adapter 10 by means of an injection molding process, a gate 49 can be located within the grip 41.

FIG. 4 shows an adapter 10 according to the invention in plan view from the side, with additional depiction of covers 60 (A), which are applied to the adapter 10 to establish an operating mode (B), and an IR spectrometer 80 located on the operational adapter 10 in a measurement position (C).

FIG. 4A shows an adapter 10 according to the invention that is not in an operating mode, since the cover 60 has not yet been applied. In this case, all openings 26, 27 are open. To establish an operating mode of the adapter 10, at least one cover 60 is applied to the adapter 10.

FIG. 4B shows an adapter from the side and therefore depicts only one side wall 33 having a covered opening 26. In this example, the opening 27 on the other side wall 33 that is not depicted can be covered by a second cover 60 (not shown).

The adapter 10 according to the invention therefore comprises not only the four abovementioned components housing entrance 16, housing exit 18, accommodator 40 and base body 20 but also at least one cover 60.

By means of the cover 60, at least one of the at least two openings 26, 27 is covered to establish an operating mode. In some embodiments, it is conceivable that only one of the at least two openings 26, 27 is covered to establish an operating mode. In this case, what should be covered is that opening 26, 27 which is located between a sensor of the IR spectrometer 80 and the adapter 10. The openings 26, 27 located between a receiver of the IR spectrometer 80 and the adapter 10 can remain open in some embodiments. As depicted advantageously in the following exemplary embodiments, both of the at least two openings 26, 27 are covered to establish an operating mode. The openings 26, 27 can then be covered by an identical cover 60. However, it is also conceivable that the different openings 26, 27 are covered by differently designed covers 60 which differ from one another in the material chosen, their shape, their thickness or other properties.

Adapter 10 and cover 60 can be in the form of one piece or two pieces. A two-piece design offers the advantage of simple and cost-effective production. Adapter 10 and cover 60 can be made of the same material or be made of different materials.

The cover 60 must be made of a material which lets radiation through. In particular, the cover 60 must be transmissive for IR radiation. The cover 60 should have a transmissivity for infrared radiation of at least about 0.5. This means that at least about 50% of the infrared radiation must be let through the cover 60 in order to allow reliable and reproducible measurement.

The cover 60 can be a film 60. The film 60 can, for example, be made of a plastic. Examples of suitable plastics include polycarbonates (PC), polymethyl methacrylates (PMMA), polystyrenes (PS) or cycloolefin copolymer (COC, Topas). For example, the film 60 can be made of PMMA Plexiglas 7N, Topas 6017-S04, PC Lexan 121 RM1, PC Makrolon 2400, PS Styrolution 124 L, Topas 8007 or COC 8007 X-04. Preferably, the film 60 is made of COC 8007 X-04.

In the present exemplary embodiment, adapter 10 and film 60 are in the form of two pieces and made of the same material. The materials of adapter 10 and film 60 chosen can also be selected independently of one another.

The film 60 has a suitable thickness which lets IR radiation through to a sufficient extent in order to allow precise and reproducible measurements. The film 60 is from about 2,000 μm to about 50 μm in thickness, preferably from about 1,000 μm to about 100 μm, particularly preferably from about 600 μm to about 100 μm, for example about 140 μm in thickness.

To establish an operating mode of the adapter 10, the at least one film 60 is applied to the adapter 10 (see FIG. 4B). The application of the film 60 to the adapter 10 can be reversible or irreversible. The film 60 is larger than the opening 26, 27. The film 60 completely covers the openings 26, 27 in operating mode. For this purpose, the film 60 is joined to at least one side wall 33 of the base body 20 at least regionally. The film 60 can be joined to the entire side wall 33 or to portions of the side wall 33. The film 60 can, for example, be joined to the inner edge 25 and/or the outer edge 23 and/or the side face 24.

In the present exemplary embodiment, one film 60 is used per opening 26 or 27. The film 60 is, for example, sufficiently large to virtually cover an entire side wall 33. In this example, the film 60 covers the opening 26, 27, the inner edge 25, the outer edge 23 and the side face 24. Only the chamfers 22 are not covered by the film (see FIG. 4B).

It is also conceivable that the film 60 is smaller and, for example, merely covers the openings 26, 27 and the inner edge 25. The film 60 can have be of any suitable size. It is also conceivable that the film 60 is larger than what is shown in FIG. 4. For example, it is conceivable that a single film 60 can cover the two openings 26, 27 at the same time. In such an embodiment, the film 60 can run from one side wall 33 over the top side 21 and/or over the bottom side 31 to the second side wall 33 (not shown).

FIG. 4C shows schematically an adapter 10 in operating mode, onto which an IR spectrometer 80 has been mounted. The IR spectrometer 80 is mounted onto the operational adapter 10 from above such that it surrounds at least the openings 26, 27 of the base body.

The IR spectrometer 80 comprises a means for emitting IR radiation, also called a sensor, a means for receiving IR radiation, also called a receiver, an analyzer for the radiation received, and optionally a cable for data transmission (not shown).

The shape of the accommodation side walls 42, 43 and the accommodation base 48 is complementary in shape to a compatible IR spectrometer 80. The IR spectrometer 80 is put over the adapter 10 in such a way that it is arranged between the accommodation side walls 42, 43 and the accommodation base 48 and substantially encloses the base body 20.

The at least one accommodation latch 44 is configured and designed to latch the IR spectrometer 80 on the adapter 10. The accommodation latch 44 is located at the top end of the accommodation side walls 42 and/or 43. The accommodation latch 44 is in the form of a projection which inwardly projects into the U-shaped accommodator 40. The accommodation latch 44 is therefore always oriented toward the base body 20. The accommodation latch 44 is, for example, integrally formed semicircularly on the accommodation side walls 42, 43. The accommodation latch 44 is, for example, about 0.5 mm in depth.

The accommodation latch 44 can extend over the entire breadth of an accommodation side wall 42, 43. To save material, it is advantageous to keep the accommodation latch 44 as small as possible. The accommodation latch 44 can, for example, not extend over the entire top end of the accommodation side wall 42, 43. The accommodation latch 44 is, for example, arranged in the middle of the top end of the accommodation side wall 42, 43.

The material of the accommodation side walls 42,43 is slightly soft. As a result, the IR spectrometer 80 can be pushed onto the adapter 10 from above with light pressure. The accommodation side walls 42, 43 yield somewhat under light pressure and allow the accommodation of the IR spectrometer 80 in the accommodator 40. When the IR spectrometer 80 is located in a measurement position, the accommodation side walls 42 43 gently spring back into their starting position.

The accommodation latch 44 can then mark the upper limit of the IR spectrometer 80. In this way, the IR spectrometer 80 can be kept in its measurement position (see FIG. 4C). It is also conceivable that the IR spectrometer 80 has a counterpart complementary in shape to the accommodation latch 44, into which the accommodation latch 44 is inserted.

The accommodation latch 44 is configured and designed such that a user pushing the IR spectrometer 80 into the accommodator 40 of the adapter 10 receives tactile and/er acoustic feedback about correct latching. Following correct latching, the IR spectrometer 80 is located on the adapter 10 such that measurement can be carried out using the IR spectrometer 80.

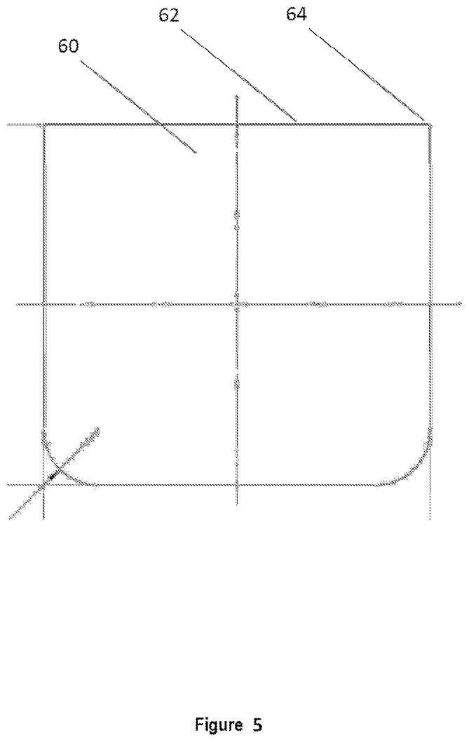

FIG. 5 shows a film 60 for the adapter 10 according to the invention. The film 60 has a polygonal shape. For example, the film 60 has a quadrangular shape. It is also conceivable that the film 60 has more than four corners.

The film 60 is, for example, quadrangular with four side lengths 62 and four corners 64. The side lengths 62 can be identical in length, and so the film 60 is square. The side lengths 62 can also differ in length, and so the film 60 is rectangular. The side lengths 62 of the film 60 have a length of from about 5 mm to about 60 mm. The side lengths 62 of the film 60 have, for example, a length of from about 10 mm to about 30 mm. For example, the side lengths 62 are from about 15 mm to about 20 mm in length. The side lengths 62 of the film 60 are straight or curved. For example, the side lengths 62 of the film 60 are straight. At least one corner 64 can be rounded. For example, two corners 64 are rounded. It is also conceivable that three or more corners 64 are rounded.

The film 60 can cover the openings 26, 27 such that the passage channel 12 is sealed in an air-tight manner. Once at least one of the two openings 26, 27 of the adapter 10 is closed, the adapter 10 is in an operating mode. Preferably, both openings 26, 27 are covered to establish an operating mode (see FIG. 4B).

The application of the film 60 to the adapter 10 can be reversible or irreversible. Adapter 10 and film 60 can be designed as disposable or reusable products. In some embodiments, adapter 10 and film 60 can be designed as disposable articles.

In alternative embodiments, it is conceivable that adapter 10 and/or film 60 are reusable. In an advantageous exemplary embodiment, the adapter 10 could be made of a sterilizable material that withstands treatment. The film 60 can be removed for the cleaning process and be respectively restored for repeated uses of the adapter 10. Therefore, the adapter 10 according to the invention offers the advantage that it can be produced cost-effectively and can nevertheless be reused—at least to some extent.

In some embodiments, the film 60 covers the openings 26, 27 such that passage of gases is possible solely from the housing entrance 16 to the housing exit 18 and vice versa. For this purpose, the film 60 is joined by adhesive bonding, heat sealing and/or mechanical latching to the side wall 33 or to portions of the side wall 33 of the base body 20 at least regionally. Heat sealing can be performed by means of the method of laser welding.

From about 20% to about 80% of the film 60 are joined to the side wall 33 or to portions of the side wall 33. Preferably, from about 30% to about 60% of the film 60 are joined to the side wall 33 or to portions of the side wall 33, for example from about 40% to about 50%.

For example, the film is joined to the inner edge 25 and/or the outer edge 23 and/or the side face 24.

For example, the film 60 is joined to the inner edge 25 and/or the outer edge 23 and/or the side face 24 by means of laser welding.

In a preferred embodiment, from about 40% to about 50% of the film 60 are heat-sealed by means of the process of laser welding to the inner edge 25 and the outer edge 23. This can achieve optimal fixation and stabilization of the film 60.

The method of laser welding offers the advantage that the film 60 is joined to the adapter 10 in a durable and tight manner. After laser welding, film 60 and adapter 10 remain fixedly joined to one another even under elevated pressure. Heat sealing is particularly durable and stable when adapter 10 and film 60 consist of the same material. However, it is also conceivable that adapter 10 and film 60 consist of different materials and are nevertheless joined to one another by means of laser welding. In that case, the use of an absorber in at least one of the two joining partners would be necessary. The process of laser welding proceeds in a rapid, simple and efficient manner. Laser welding makes it possible to transfer the adapter 10 into an operating mode in a simple, qualified and validated process.

The film 60 can be cut and/or punched into the required or desired shape. This manner of production is particularly effective and cost-effective. Slight deviations in the size of the film 60 do not have any substantial influence on the quality of the adapter 10 in operating mode. Therefore, production of the operational adapter 10 can be accomplished in a simple and cost-effective manner without appreciable rejects arising.

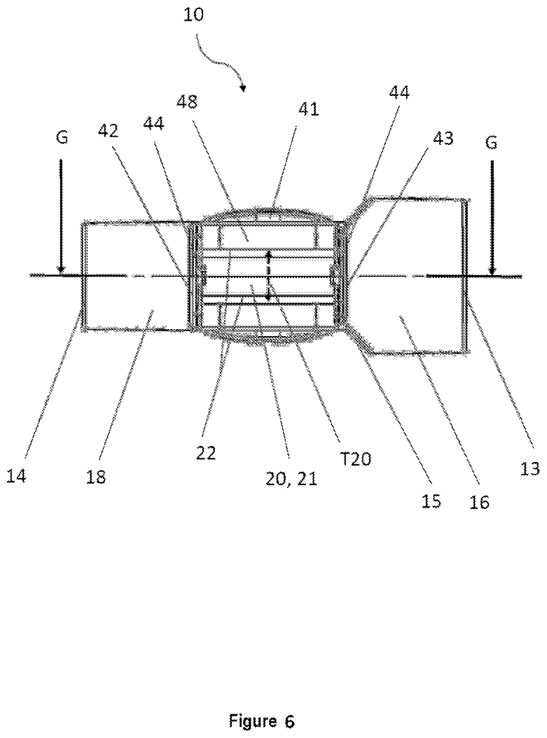

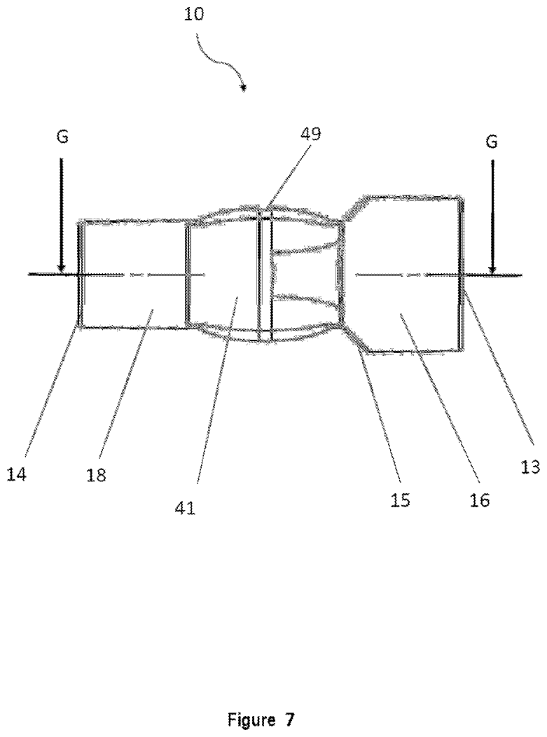

FIG. 6 shows an adapter 10 according to the invention in plan view from the top and FIG. 7 shows an adapter 10 according to the invention in plan view from the bottom. FIG. 6 makes it clear that the depth of the base body T20 is, as described above, narrower than the accommodation side walls 42, 43 and the accommodation base 48. Furthermore, it is clear that the accommodation latches 44 are integrally formed on the accommodation side walls 42, 43 in the direction of the base body 20 and are in the form of a slight projection.

Moreover, it is clear from FIG. 6 that the housing entrance 16 has a larger diameter than the housing exit 18. So that the housing entrance 16 having a larger cross section can connect to the base body 20, the housing entrance 16 has at least one flattening 15 on the side opposite the inlet opening 13.

Furthermore, it is shown that, in this exemplary embodiment, the grip 41 is broader than the accommodator 40. The broadened design of the grip 41 facilitates gripping of the adapter 10. A grip 41 of a different design is also possible.

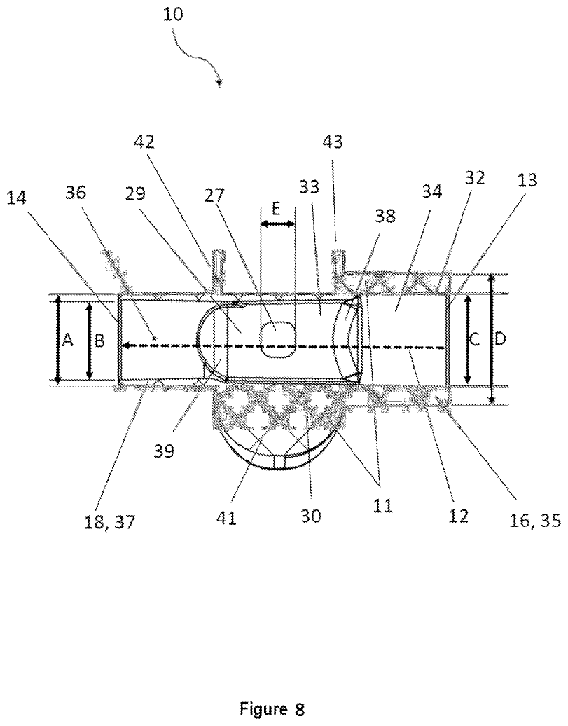

Sectional plane G for FIG. 8, which will be described below, can be seen in FIGS. 6 and 7.

FIG. 8 shows a longitudinal section according to line G-G through an adapter 10 according to the invention.

It is clear that the passage channel 12 is delimited by the housing 11. The housing 11 encloses the adapter 10 in its entire length L10 (see FIG. 1). The passage channel 12 therefore extends through the entire length L10 of the housing 11.

The housing 11 comprises a housing entrance wall 35, a base body wall 30 and a housing exit wall 37, which delimit the passage channel 12.

The housing entrance wall 35 encloses the elongated hollow body of the housing entrance 16, which is circular for example. The housing entrance wall 35 encloses a housing entrance interior 34. The housing entrance 16 has, for example, a constant inner diameter which corresponds to an inner diameter C of the inlet opening 13. The inner diameter can also become progressively larger or smaller.

The housing exit wall 37 encloses the elongated hollow body of the housing exit 18, which is circular for example. The housing exit wall 37 encloses a housing exit interior 36. The housing exit 18 has, for example, a constant inner diameter which corresponds to an inner diameter B of the outlet opening 14. The inner diameter can also become progressively larger or smaller.

The base body wall 30 encloses the hollow body of the base body 20, which is, for example, in the form of a square tube having plane-parallel faces. The base body wall 30 encloses a base body interior 29. The base body 20 is, for example, constant in its height H20 and its depth T20, and so the passage channel 12 is constant in the region of the base body 20.

The passage channel 12 extends from the inlet opening 13 to the outlet opening 14 via the housing entrance interior 34, the base body interior 29 and the housing exit interior 36. The inner diameter of the passage channel 12 can be constant. In the exemplary embodiment depicted, the diameter of the passage channel 12 is, for example, not constant.

For example, an inner diameter C of the inlet opening 13 is larger than an inner diameter B of the outlet opening 14. The inner diameter C of the inlet opening 13 is within a range between 25 and 5 mm. The inner diameter C of the inlet opening 13 is, for example, 15.56 mm±0.05. The inner diameter B of the outlet opening 14 is within a range from about 25 mm to about 5 mm. The inner diameter B of the outlet opening 14 is, for example, 12.4 mm±0.05.

The wall thickness of the housing entrance 16 and the housing exit 18 can be identical or differ greatly. In the present exemplary embodiment, the wall thickness of the housing entrance wall 35 is greater than the wall thickness of the housing exit wall 37. This means that the outer diameter D of the inlet opening 13 is larger than the outer diameter A of the outlet opening 14.

The outer diameter D of the inlet opening 13 is within a range of from about 35 mm to about 5 mm. The outer diameter D of the inlet opening 13 is, for example, 22.01 mm±0.05. The outer diameter A of the outlet opening 14 is within a range from about 35 mm to about 5 mm. The outer diameter of the outlet opening A is, for example, 15.23 mm±0.05.

The passage channel 12 can have different cross sections owing to the respective events in the different components. In some embodiments, the cross section of the passage channel 12 can also be constant.

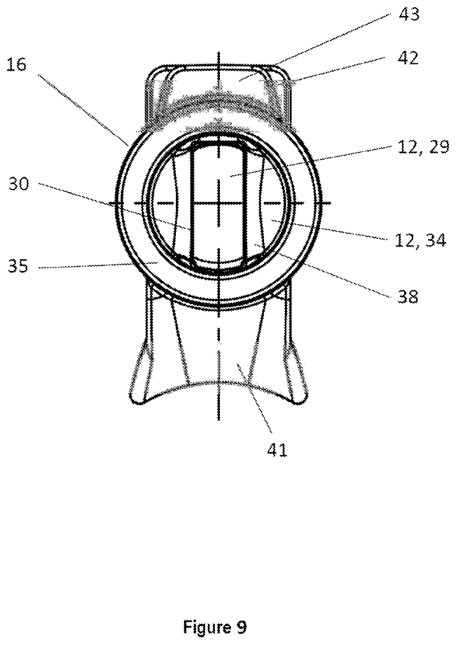

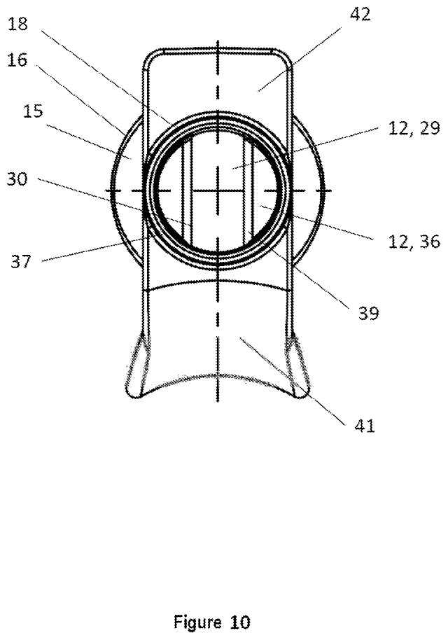

In the present exemplary embodiment, the passage channel 12 has a round cross section, for example, in the region of the housing entrance 16 and the housing exit 18 (see below, FIG. 9) and a quadrangular cross section, for example, in the region of the base body 20 (see below, FIG. 10).

The passage channel 12 runs gradually from the housing entrance interior 34 to the base body interior 29 via an entrance transition point 38. The entrance transition point 38 is funnel-shaped, and so there is a gradual narrowing and change in cross section of the passage channel 12. The funnel-shaped course of the entrance transition point 38 means that the flow property of the passage channel 12 is optimized and that turbulences and/or disturbances of the gas flow are avoided.

The passage channel 12 runs from the base body interior 29 to the housing exit interior 36 via an exit transition point 39. The exit transition point 39 can be funnel-shaped. In the exemplary embodiment depicted, the exit transition point is not funnel-shaped. Between the angular cross section of the base body 20 and the circular cross section of the housing exit 18, no funnel-shaped narrowing is provided, for example.

The longitudinal section according to line G-G that is depicted in FIG. 8 further shows that the housing inner wall 32 is smooth. A smooth housing inner wall 32 reduces turbulences or disturbances of the gas flow. It should be emphasized that the housing inner wall 32 is also smooth in the region of the side wall 33. There is no profiling in the interior of the base body 20. The housing inner wall 32 is breached by at least two lateral openings 26, 27, only one of which is depictable in the longitudinal section. The opening 26, 27 is, as described herein above, in the form of a polygon, for example as a square opening 26, 27 having rounded corners.

The square opening 26, 27 is, in terms of its height and breadth E, not more than about 15 mm in size, preferably less than about 10 mm. In an exemplary embodiment, the height and breadth E is within a range from about 8 mm to about 1 mm. The size of the opening E is, for example, 5.88 mm±0.20.

FIG. 9 shows an adapter 10 according to the invention in plan view from the housing entrance side 16. It is clear from FIG. 9 that the housing entrance 16 in the exemplary embodiment depicted has a circular cross section. The passage channel 12 extends through the circular cavity of the housing entrance interior 34.

In this exemplary embodiment, the entrance transition point 38 is rounded. The entrance transition point 38 forms a funnel-shaped course of the passage channel 12 from the housing entrance interior 34 to the base body interior 29. Thus, the cross section of the passage channel 12 is circular in the housing entrance interior 34 and quadrangular in the base body interior 29.

Furthermore, FIG. 9 makes it clear that the accommodation side wall 43 tapers toward the top, whereas the accommodation side wall 42 remains constant in width. This solution allows compatibility of the adapter 10 with CO₂ sensors from different manufacturers.

FIG. 10 shows an adapter 10 according to the invention in plan view from the housing exit side 18. It is clear from FIG. 10 that the housing exit 18 in the exemplary embodiment depicted has a circular cross section. Furthermore, it is clear that the housing exit 18 in this exemplary embodiment has a smaller diameter than the housing entrance 16. The passage channel 12 extends through the circular cavity of the housing exit interior 36. Furthermore, the passage channel 12 extends through the quadrangular cavity of the base body interior 29. In the exemplary embodiment depicted, the exit transition point 39 is not rounded or funnel-shaped.

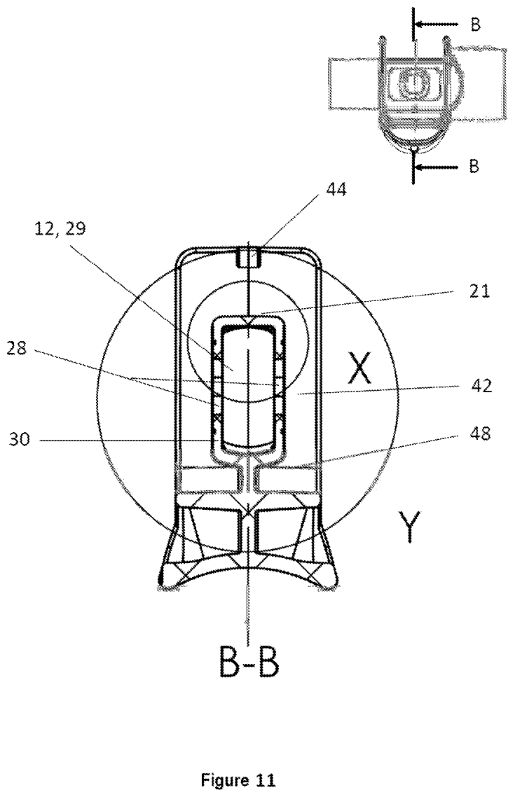

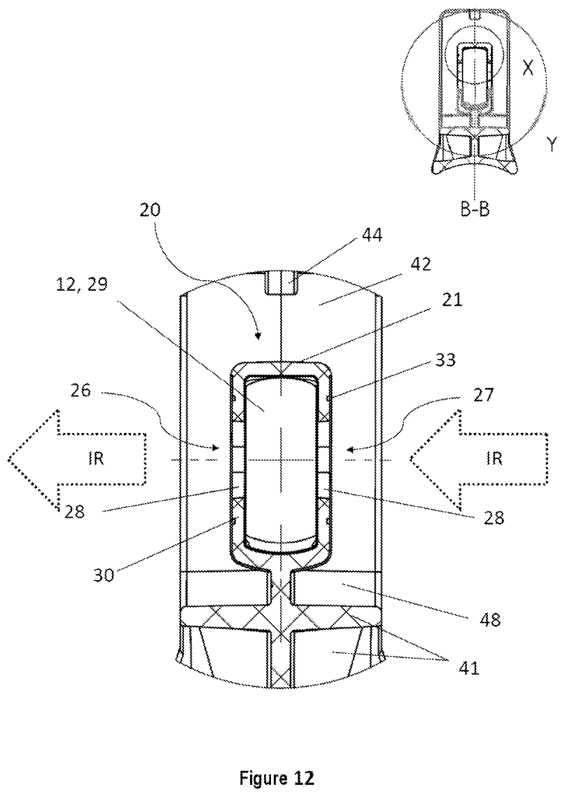

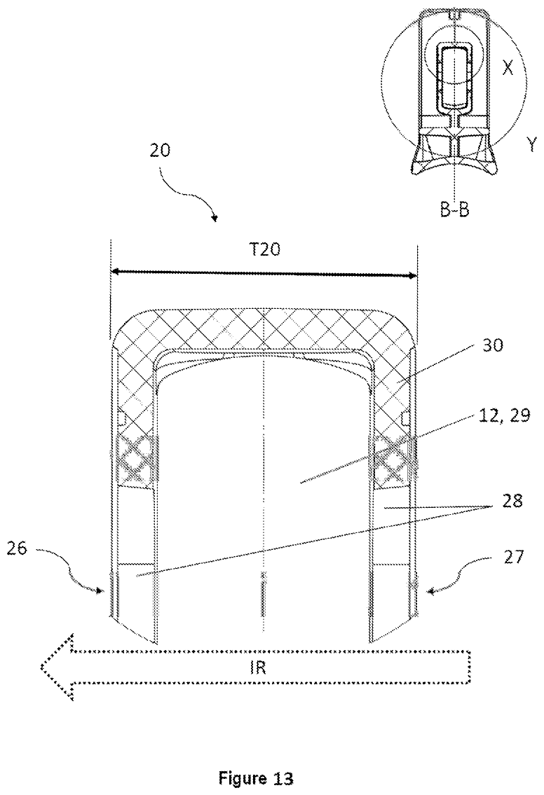

FIG. 11 shows a cross section according to line B-B, which essentially runs through the base body 20 of an adapter 10 according to the invention. FIG. 12 shows a detailed view of the section according to line B-B in the region Y. FIG. 13 shows a detailed view of the section according to line B-B in the region X.

FIGS. 11 to 13 show in detail, by way of example, how the region in which measurement can be carried out using the IR spectrometer 80, which is not shown, can be designed.

FIG. 12 shows that the base body 20 has substantially plane-parallel side walls 33. The base body wall 30 is breached by at least two openings 26, 27. These are edged by the reveals 28. It is through the two openings 26, 27 that radiation, for example IR radiation, can be directed through the base body 20 such that the IR radiation intersects the base body interior 29 and thus also the passage channel 12. The IR radiation can be conducted into the base body interior 29 through the opening 27 and conducted out of the base body interior 29 through the opening 26. The IR radiation can also run in the reverse direction, i.e., from the opening 26 to the opening 27. Because the IR radiation intersects the passage channel 12, gases, for example breathing gases, which are present in the passage channel 12 and/or are guided therethrough, can be spectrometrically analyzed.

FIG. 13 shows a detailed view of the section according to line B-B in the region X. The base body wall 30 is breached by the two openings 26, 27 which are edged by the reveals 28 (partial depiction). What is schematically indicated is how the IR radiation intersects the base body 20 in its entire depth T20. Here, the IR radiation also crosses the breathing gas present in the passage channel 12.

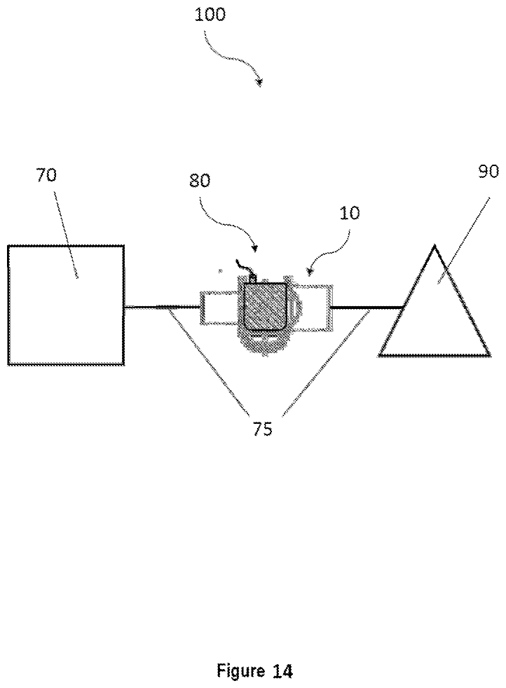

The adapter 10 according to the invention is, for example, suitable for use with a system 100 for analysis of a stream of breathing gas, i.e., for use with a ventilator 70 and/or a patient interface 90 and an IR spectrometer. In the system 100, use can be made of one or more adapters 10, the at least one adapter 10 being gaseously connected to the patient interface 90 and/or the ventilator 70. FIG. 14 shows, by way of example, a schematic overall view of a system 100 comprising an adapter 10 according to the invention having a mounted IR spectrometer 80 that is gaseously connected to a ventilator 70 and a patient interface 90 via a line 75.

The adapter 10 according to the invention is usable with a patient interface 90. Patient interface 90 is to be understood to mean any peripheral device designed for interaction with a living being. In particular, the patient interface 90 is designed for therapeutic or diagnostic purposes in connection with the adapter 10 and/or with the ventilator 70. The patient interface 90 can be in the form of a mask. Said mask can be a full-face mask, i.e., surrounding the nose and mouth, or a nasal mask, i.e., a mask only surrounding the nose. Tracheal tubes or cannulas and so-called nasal cannulas can be used as a patient interface 90, too. In some cases, the patient interface 90 can also be a simple mouthpiece, for example a tube or hose, through which the living being exhales and/or inhales.

The adapter 10 can be arranged in and/or on the patient interface 90. In some embodiments, the adapter 10 can be directly integrated into the patient interface 90. In other embodiments, adapter 10 and patient interface 90 can be directly couplable to one another. Adapter 10 and patient interface 90 can also be connected to one another via at least one line 75.

The adapter 10 according to the invention is usable either with a ventilator 70 or without a ventilator 70. A ventilator 70 is to be understood to mean devices which assist a user or patient with natural respiration and/or undertake the ventilation of a user or patient and/or is used for respiratory therapy and/or affects the respiration of a user or patient in another way. By way of example, but without being an exhaustive list, these include CPAP and BiPAP machines, anesthetic machines, respiratory therapy devices, clinical, outpatient or emergency ventilators, high-flow therapy devices and cough machines.

Ventilators 70 can also be understood to mean diagnostic devices for ventilation. Said diagnostic devices can generally be used to measure medical, physiological and/or respiration-based parameters of a living being. These also include devices which can measure and optionally process medical parameters of patients in combination with respiration or only in relation to respiration.

The adapter 10 according to the invention can be arranged in and/or on the ventilator 70. In some embodiments, the adapter 10 can be directly integrated into the ventilator 70. In other embodiments, adapter 10 and ventilator 70 can be directly couplable to one another. Adapter 10 and ventilator 70 can also be connected to one another via at least one line 75.

Patient interface 90 and/or ventilator 70 and/or adapter 10 are preferably connected to one another via at least one line 75. The line 75 is in the form of a gas line which connects the individual components of the system 100 to one another. The line 75 is preferably designed such that no unwanted leakage occurs. Furthermore, the connection is preferably flexible and/or rotatable. The line 75 can, for example, be in the form of an elastic tube and/or hose and/or hose system.

The adapter 10 according to the invention can be arranged between a patient interface 90 and a ventilator 70, for example, for analysis purposes, and so the patient interface 90 is gaseously connected to the ventilator 70 via the adapter 10 (see FIG. 14).

For analysis of the breathing gas, the adapter 10 according to the invention is, for example, used in conjunction with an IR spectrometer 80. The IR spectrometer 80 can emit IR radiation by means of a sensor. Said IR radiation is, as described in detail herein, conducted through the adapter 10 according to the invention, the IR radiation intersecting the breathing gas. The IR radiation is then received by a receiver. An analyzer can analyze the IR radiation received and optionally transmit data to a data storage medium via a cable for data transmission.

To sum up, the present invention provides the following items:
1. An adapter which is suitable for use with spectrometers for the analysis of gases and comprises a housing which comprises a base body, an accommodator for accommodation of a spectrometer, a housing entrance and a housing exit, the housing entrance comprising an inlet opening and the housing exit comprising an outlet opening, a passage channel being arranged between the inlet opening and the outlet opening, and the base body being arranged between the housing entrance and the housing exit and having at least two openings which are arranged on two sides of the passage channel on opposite sides.
2. The adapter of item 1, wherein the base body comprises two side walls which each have an opening through which radiation can be directed such that the radiation intersects the passage channel, the radiation being infrared radiation.
3. The adapter of any one of the preceding items, wherein the adapter has a maximum length L10, wherein the passage channel 12 extends from the inlet opening to the outlet opening over the maximum length L10 of the adapter 10, the passage channel being designed and configured to guide breathing gases.
4. The adapter of any one of the preceding items, wherein the at least two openings of the base body are a polygon, preferably a quadrangle, in terms of their basic shape.
5. The adapter of any one of the preceding items, wherein at least one of the at least two openings of the base body is covered by at least one cover to establish an operating mode.
6. The adapter of item 5, wherein adapter and cover are in the form of one piece or two pieces, adapter and cover being made of the same material or of different materials.
7. The adapter of any one of items 5 and 6, wherein adapter and cover are in the form of two pieces and made of the same material.
8. The adapter of any one of items 5 to 7, wherein adapter and cover are made of a plastic, preferably a thermoplastic.
9. The adapter of any one of items 5 to 8, wherein a material of the adapter and of the cover comprises one or more of polycarbonates (PC), polymethyl methacrylates (PMMA), polystyrenes (PS), cycloolefin copolymers (COC, Topas).
10. The adapter of any one of items 5 to 9, wherein the adapter and the cover are made of one or more of PMMA Plexiglas 7N, Topas 6017-S04, PC Lexan 121 RM1, PC Makrolon 2400, PS Styrolution 124 L, Topas 8007 or COC 8007 X-04, preferably of COC or PMMA.
11. The adapter of any one of items 5 to 10, wherein the cover is transmissive for radiation, especially IR radiation.
12. The adapter of any one of items 5 to 11, wherein the cover 60 has a transmissivity for infrared radiation of at least 0.5.
13. The adapter of any one of items 5 to 12, wherein the cover is present as a film.
14. The adapter of item 13, wherein the film has a thickness of from 2,000 μm to 50 μm, preferably of from 1000 μm to 100 μm, particularly preferably of from 600 μm to 100 μm, for example 140 μm.
15. The adapter of any one of items 13 and 14, wherein the film is larger than the at least to openings of the base body.
16. The adapter of any one of items 13 to 15, wherein the base body comprises two side walls which each comprise an opening, an inner edge, an outer edge and a side face and wherein the film is applied to the side walls at least regionally and completely covers at least one of the at least two openings of the base body to establish an operating mode of the adapter.
17. The adapter of any one of items 13 to 16, wherein the film covers the at least two openings of the base body such that the passage channel is sealed in an air-tight manner and passage of gases is solely possible from the housing entrance to the housing exit and vice versa.
18. The adapter of any one of items 16 and 17, wherein the film is joined to the side wall or portions of the side wall by adhesive bonding, heat sealing and/or mechanical latching.
19. The adapter of any one of items 16 to 18, wherein from 20% to 80% of the film are joined to the side wall 33 or to portions of the side wall, preferably from 30% to 60%, particularly preferably from 40% to 50%.
20. The adapter of any one of items 16 to 19, wherein the film is joined to the inner edge and/or the outer edge and/or the side face at least regionally.
21. The adapter of any one of items 16 to 20, wherein the film and the inner edge and/or the outer edge and/or the side face are joined by means of laser welding.
22. The adapter of any one of items 13 and 21, wherein the film has a polygonal shape, preferably a quadrangular shape.
23. The adapter of any one of items 13 to 22, wherein the film comprises side lengths, the side lengths of the film having a length of from 5 mm to 60 mm, preferably from 10 mm to 30 mm, particularly preferably from 15 to 20 mm.
24. The adapter of any one of the preceding items, wherein the accommodator comprises an accommodation base and two accommodation side walls and the accommodator is configured and designed to accommodate a shape-complementary IR spectrometer between the two accommodation side walls and the accommodation base such that at least the at least two openings of the base body are enclosed by the IR spectrometer.
25. A system suitable for analysis of a stream of breathing gas, wherein the system comprises at least a ventilator and/or a patient interface and an IR spectrometer, and wherein the system further comprises at least one adapter according to any one of the preceding items, the adapter being gaseously connected to the ventilator and/or the patient interface and the IR spectrometer enclosing the adapter at least regionally such that IR irradiation intersects the stream of breathing gas.

Although the present invention has been described in detail on the basis of exemplary embodiments, it is self-evident to a person skilled in the art that the invention is not restricted to said exemplary embodiments. On the contrary, modifications involving omission of individual features or realization of different combinations of the individual features described are possible, provided that there is no departure from the scope of protection of the appended claims. The present disclosure includes all combinations of the individual features presented.

### LIST OF REFERENCE SIGNS

10 Adapter

H10 Height of the adapter

L10 Length of the adapter

11 Housing

12 Passage channel

13 Inlet opening

14 Outlet opening

15 Flattening

16 Housing entrance

18 Housing exit

20 Base body

H20 Height of the base body

L20 Length of the base body

T20 Depth of the base body

21 Top side

22 Chamfer

23 Outer edge

24 Side face

25 Inner edge

26 Opening

27 Opening

28 Reveal

29 Base body interior

30 Base body wall

31 Bottom side

32 Housing inner wall

33 Side wall

34 Housing entrance interior

35 Housing entrance wall

36 Housing exit interior

37 Housing exit wall

38 Entrance transition point

39 Exit transition point

40 Accommodator

41 Grip

42 Accommodation side wall

43 Accommodation side wall

44 Accommodation latch

48 Accommodation base

49 Gate

60 Cover/film

62 Side length

64 Corner

70 Ventilator

75 Line

80 IR spectrometer

90 Patient interface

100 System

A Outer diameter of the outlet opening

B Inner diameter of the outlet opening

C Inner diameter of the inlet opening

D Outer diameter of the inlet opening

E Size of opening

O Top

U Bottom

What is claimed is:

1. An adapter suitable for use with spectrometers for the analysis of gases, wherein the adapter comprises a housing which comprises a base body, an accommodator for accommodation of a spectrometer, a housing entrance and a housing exit, the housing entrance comprising an inlet opening and the housing exit comprising an outlet opening, a passage channel being arranged between the inlet opening and the outlet opening, and the base body being arranged between the housing entrance and the housing exit and having at least two openings which are arranged on two sides of the passage channel on opposite sides.

2. The adapter of claim 1, wherein the base body comprises two side walls which each have an opening through which radiation can be directed such that the radiation intersects the passage channel, the radiation being infrared radiation.

3. The adapter of claim 1, wherein the adapter has a maximum length L10 and the passage channel extends from the inlet opening to the outlet opening over the maximum length L10 of the adapter 10, the passage channel being designed and configured to guide breathing gases.

4. The adapter of claim 1, wherein the at least two openings of the base body are a polygon, in terms of their basic shape.

5. The adapter of claim 1, wherein at least one of the at least two openings of the base body is covered by at least one cover to establish an operating mode.

6. The adapter of claim 5, wherein adapter and cover are in the form of two pieces and made of the same material.

7. The adapter of claim 5, wherein adapter and cover are made of a plastic.

8. The adapter of claim 5, wherein a material of the adapter and of the cover comprises one or more of polycarbonates (PC), polymethyl methacrylates (PMMA), polystyrenes (PS), cycloolefin copolymers (COC, Topas).

9. The adapter of claim 5, wherein the cover is transmissive for radiation.

10. The adapter of claim 5, wherein the cover 60 has a transmissivity for infrared radiation of at least 0.5.

11. The adapter of claim 5, wherein the cover is present as a film.

12. The adapter of claim 11, wherein the film has a thickness of from 2,000 μm to 50 μm.

13. The adapter of claim 11, wherein the film is larger than the at least to openings of the base body.

14. The adapter of claim 11, wherein the base body comprises two side walls which each comprise an opening, an inner edge, an outer edge and a side face and wherein the film is applied to the side walls at least regionally and completely covers at least one of the at least two openings of the base body to establish an operating mode of the adapter.

15. The adapter of claim 14, wherein the film covers the at least two openings of the base body such that the passage channel is sealed in an air-tight manner and passage of gases is solely possible from the housing entrance to the housing exit and vice versa.

16. The adapter of claim 14, wherein the film is joined to the side wall or portions of the side wall by adhesive bonding, heat sealing and/or mechanical latching.

17. The adapter of claim 11, wherein the film has a polygonal shape.

18. The adapter of claim 11, wherein the film comprises side lengths, the side lengths of the film having a length of from 5 mm to 60 mm.

19. The adapter of claim 1, wherein the accommodator comprises an accommodation base and two accommodation side walls and the accommodator is configured and designed to accommodate a shape-complementary IR spectrometer between the two accommodation side walls and the accommodation base such that at least the at least two openings of the base body are enclosed by the IR spectrometer.

20. A system suitable for analysis of a stream of breathing gas, wherein the system comprises at least a ventilator and/or a patient interface and an IR spectrometer, and wherein the system further comprises at least one adapter according to claim 1, the adapter being gaseously connected to the ventilator and/or the patient interface and the IR spectrometer enclosing the adapter at least regionally such that IR irradiation intersects the stream of breathing gas.
